SEQUENCE LISTING (2 sequences)

SEQ ID NO: 1 - protein, 40 aa, Artificial Sequence
  Synthetic construct
GFGCNGPWNEDDLRCHNHCKSIKGYKGGYCAKGGFVCKCY

SEQ ID NO: 2 - protein, 40 aa, Pseudoplectania nigrella
  mat_peptide (1)..(40)
GFGCNGPWDEDDMQCHNHCKSIKGYKGGYCAKGGFVCKCY

(19) United States
(12) Patent Application Publication
(10) Pub. No.: US 2010/0105602 A1
(43) Pub. Date: Apr. 29, 2010

(54) TREATMENT OF INTRACELLULAR BACTERIAL INFECTIONS

(75) Inventor: Karoline Sidelmann Brinch, Copenhagen (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/604,443

(22) Filed: Oct. 23, 2009

Related U.S. Application Data
(60) Provisional application No. 61/109,233, filed on Oct. 29, 2008.
(60) Provisional application No. 61/169,430, filed on Apr. 15, 2009.

(30) Foreign Application Priority Data
Oct. 24, 2008 (EP) 08167489.7
Mar. 31, 2009 (EP) 09156992.1

(51) Int. Cl. A61K 38/02; C07K 2/00; A61P 37/04; A61P 31/04

(52) U.S. Cl. 514/2; 530/300

(57) ABSTRACT

The present invention relates to methods for treating intracellular bacterial infections with defensin polypeptides.

### CROSS-REFERENCE TO RELATED APPLICATIONS

This application claims priority or the benefit under 35 U.S.C. 119 of European application nos. 08167489.7 and EP 09156992.1 filed Oct. 24, 2008 and Mar. 31, 2009, respectively, and U.S. provisional application Nos. 61/109,233 and 61/169,430 filed Oct. 29, 2008 and Apr. 15, 2009, respectively, the contents of which are fully incorporated herein by reference.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to the treatment of intracellular bacterial infections in phagocytic cells with defensin polypeptides.

2. Description of Related Art

Staphylococcus aureus is the major causative agent of numerous community and hospital-acquired infections ranging from minor infections such as dermatitis or wound infections to critical septicaemia-related diseases, including arthritis, endocarditis, pneumonia and meningitis.

Treatment of staphylococcal diseases is often challenging since the infection often persist or recur even after prolonged treatment with antimicrobial agents.

Several aspects of bacterial pathogenesis may be involved in the persistence of staphylococcal infections. An important feature reported by several authors is the bacteria's ability to invade the phagocytes and survive inside the cells.

Intracellular accumulation of the bacteria complicates the use of antimicrobial agents since intracellular activity depend on several factors such as the drugs' ability to penetrate and accumulate in the cell and drug metabolism and distribution inside the cells. Many antibiotic agents cannot enter the phagocytes and furthermore, studies have shown that the antimicrobial activity and the bacteria's responsiveness to therapy are often impaired in drugs capable of cell-penetration.

Several authors have described the in vitro activity of antibiotics against intracellular S. aureus, but many reports yield contradictory results. Seral et al. showed that intracellular concentration and accumulation was only partially predictive of activity and it is still not clear which parameters determine an antibiotics' intracellular activity.

Gresham et al. showed that polymorph nuclear neutrophils (PMNs) infected with S. aureus contained sufficient amounts of viable intracellular bacteria to cause infection by intraperitoneal injection of these cells in healthy mice. Moreover they demonstrated that limiting the migration of PMNs to the infection site lead to an enhanced host defense. These data could indicate that intracellular survival of the bacteria is one of the key players in the pathogenesis of infections with S. aureus.

Many drugs, including commonly used glycopeptides such as Vancomycin, have poor intracellular effect against S. aureus. Additionally a rise in incidence of methicillin-resistant (MRSA) S. aureus and emergence of multidrug-resistant strains has further complicated the therapy of these infection. Therefore the need of novel compounds against these types of infections is increasing.

Plectasin is a defensin derived from the saprophytic ascomycete, Pseudoplectania nigrella. This antimicrobial peptide has shown potent antimicrobial effect both in vitro and in vivo against various gram-positive bacteria, including strains of Staphylococcus aureus. Furthermore it has a novel mode of action on the bacteria compared to commonly used anti-staphylococcal compounds. However, this or any other novel antimicrobial peptide has never, to our knowledge been tested in models of intracellular infection. Should a new drug, such as Plectasin, possess the ability to enter the cell and kill intracellular bacteria, this would be an advantage in future therapy of staphylococcal infections.

Numerous in vitro models using either human or animal cell lines exist for studies of intracellular antimicrobial activity. However, only few studies have been performed in animals.

The present invention relates to antibacterial effect against staphylococci residing inside human or animal phagocytic cells (phagocytes), such as macrophages, by administration of a defensin.

### SUMMARY OF THE INVENTION

We have now found that certain defensin polypeptides can be used for treatment of intracellular bacterial infections in phagocytic cells (phagocytes), such as macrophages.

Accordingly, in a first aspect, the present invention provides use of a polypeptide having antibacterial activity, which comprises an amino acid sequence having at least 80% identity to the amino acid sequence of SEQ ID NO: 1, for manufacturing a medicament for therapeutic treatment of an intracellular bacterial infection in a phagocytic cell, such as a macrophage.

In an embodiment, the bacterial infection is a staphylococcal infection.

In a second aspect, the invention provides a method for the treatment of an intracellular bacterial infection in a phagocytic cell, such as macrophage, comprising administering to the human or animal in need of such treatment an antibacterial polypeptide, which comprises an amino acid sequence having at least 90% identity to the amino acid sequence of SEQ ID NO: 1, in an effective amount for the treatment of the intracellular bacterial infection in a phagocytic cell.

In an embodiment, the treatment includes a first dose of the polypeptide of at least 17 mg/kg.

A polypeptide for use according to the present invention or for treating intracellular bacterial infections in phagocytic cells, such as macrophages, according to the present invention is designated hereinafter as “polypeptide(s) of (according to) the present invention”.

### DETAILED DESCRIPTION OF THE INVENTION

### Definitions

Antibacterial activity: The term “antibacterial activity” is defined herein as an activity which is capable of killing or inhibiting growth of bacterial cells. In the context of the present invention the term “antibacterial” is intended to mean that there is a bactericidal and/or a bacteriostatic effect; wherein the term “bactericidal” is to be understood as capable of killing bacterial cells; and wherein the term “bacteriostatic” is to be understood as capable of inhibiting bacterial growth. When growth of bacterial cells is inhibited, the cells are in a non-growing state, i.e., they are not able to propagate.

In a preferred embodiment, the term “antibacterial activity” is defined as bactericidal and/or bacteriostatic activity against Streptococci, preferably Streptococcus pneumoniae, or Staphylococci, preferably Staphylococcus aureus.

For purposes of the present invention, antibacterial activity may be determined according to the procedure described by Lehrer et al., 1991, Journal of Immunological Methods, 137(2): 167-174. Alternatively, antibacterial activity may be determined according to the NCCLS guidelines from CLSI (Clinical and Laboratory Standards Institute; formerly known as National Committee for Clinical and Laboratory Standards).

Compounds having antibacterial activity may be capable of reducing the number of living cells of Streptococcus pneumoniae (ATCC 49619) to 1/100 after 24 hours (preferably after 16 hours, more preferably after 8 hours, most preferably after 4 hour, and in particular after 2 hours) incubation at 37° C. in a relevant microbial growth substrate at a concentration of 500 micrograms/mL; preferably at a concentration of 250 micrograms/mL; more preferably at a concentration of 100 micrograms/mL; even more preferably at a concentration of 50 micrograms/mL; most preferably at a concentration of 25 micrograms/mL; and in particular at a concentration of 10 micrograms/mL of the polypeptides having antimicrobial activity.

Compounds having antibacterial activity may also be capable of inhibiting the outgrowth of Streptococcus pneumoniae (ATCC 49619) for 8 hours at 37° C. in a relevant microbial growth substrate, when added in a concentration of 500 micrograms/mL; preferably when added in a concentration of 250 micrograms/mL; more preferably when added in a concentration of 100 micrograms/mL; even more preferably when added in a concentration of 50 micrograms/mL; most preferably when added in a concentration of 10 micrograms/mL; and in particular when added in a concentration of 5 micrograms/mL.

The polypeptides of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the antimicrobial activity of the polypeptide consisting of the amino acid sequence of SEQ ID NO: 1.

Defensin: The term “defensin” as used herein refers to polypeptides recognized by a person skilled in the art as belonging to the defensin class of antimicrobial peptides. To determine if a polypeptide is a defensin according to the invention, the amino acid sequence is preferably compared with the hidden markov model profiles (HMM profiles) of the PFAM database by using the freely available HMMER software package (see Example 3).

The PFAM defensin families include Defensin_{—}1 or “Mammalian defensin” (accession no. PF00323), Defensin_{—}2 or “Arthropod defensin” (accession no. PF01097), Defensin_beta or “Beta Defensin” (accession no. PF00711), Defensin_propep or “Defensin propeptide” (accession no. PF00879) and Gamma-thionin or “Gamma-thionins family” (accession no. PF00304).

The defensins may belong to the alpha-defensin class, the beta-defensin class, the theta-defensin class, the insect or arthropod defensin classes, or the plant defensin class. The defensins from each of these classes share common structural features, such as the cycteine pattern. But it is important to note that the class affiliation does not reveal the source of the defensins. For example, a defensin from a fungus may be affiliated to the insect defensin class.

The defensin shown as SEQ ID NO: 1 is a synthetic defensin derived from Plectasin (see WO 03/044049), which is affiliated with the insect defensin class. Plectasin is shown in SEQ ID NO: 2.

In an embodiment, the amino acid sequence of a defensin according to the invention comprises 4, 5, 6, 7, or 8 cysteine residues, preferably 4, 5, or 6 cysteine residues, more preferably 4 or 6 cysteine residues, and most preferably 6 cysteine residues.

The defensins may also be synthetic defensins sharing the characteristic features of any of the defensin classes.

Examples of such defensins include, but are not limited to, α-Defensin HNP-1 (human neutrophil peptide) HNP-2 and HNP-3; β-Defensin-12, Drosomycin, Heliomicin, γ1-purothionin, Insect defensin A, and the defensins disclosed in PCT applications WO 99/53053, WO 02/06324, WO 02/085934, WO 03/044049, WO 2006/131504, WO 2006/050737 and WO 2006/053565.

Isolated polypeptide: The term “isolated variant” or “isolated polypeptide” as used herein refers to a variant or a polypeptide that is isolated from a source. In one aspect, the variant or polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptides of the present invention are preferably in a substantially pure form. This can be accomplished, for example, by preparing the polypeptide by well-known recombinant methods or by classical purification methods.

Identity: The relatedness between two amino acid sequences or between two nucleotide sequences is described by the parameter “identity”.

For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends in Genetics 16: 276-277; emboss.org), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the degree of identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra; emboss.org), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

Allelic variant: The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Modification: The term “modification” means herein any chemical modification of the polypeptide consisting of the amino acid sequence of SEQ ID NO: 1 as well as genetic manipulation of the DNA encoding that polypeptide. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus.

### Polypeptides Having Antibacterial Activity

In a first aspect, the present invention relates to isolated polypeptides having an amino acid sequence which has a degree of identity to SEQ ID NO: 1 (i.e., the mature polypeptides) of at least 80%, preferably at least 85%, more preferably at least 90%, most preferably at least 95%, and in particular at least 97%, which have antibacterial activity (hereinafter “homologous polypeptides”). In a preferred aspect, the homologous polypeptides have an amino acid sequence which differs by at the most six amino acids, preferably by at the most five amino acids, more preferably by at the most four amino acids, even more preferably by at the most three amino acids, most preferably by at the most two amino acids, and in particular by one amino acid from the amino acid sequence of SEQ ID NO: 1.

A polypeptide of the present invention preferably comprises the amino acid sequence of SEQ ID NO: 1 or an allelic variant thereof. In a preferred aspect, a polypeptide comprises the amino acid sequence of SEQ ID NO: 1. In another preferred aspect, a polypeptide consists of the amino acid sequence of SEQ ID NO: 1 or an allelic variant thereof. In another preferred aspect, a polypeptide consists of the amino acid sequence of SEQ ID NO: 1.

Preferably, amino acid changes are of a minor nature, that is conservative amino acid substitutions or insertions that do not significantly affect the folding and/or activity of the polypeptide; single deletions; small amino- or carboxyl-terminal extensions; a small linker peptide of up to about 20-25 residues; or a small extension that facilitates purification by changing net charge or another function, such as a poly-histidine tag, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In addition to the 20 standard amino acids, non-standard amino acids (such as 4-hydroxyproline, 6-N-methyl lysine, 2-aminoisobutyric acid, isovaline, and alpha-methyl serine) may be substituted for amino acid residues of a wild-type polypeptide. A limited number of non-conservative amino acids, amino acids that are not encoded by the genetic code, and unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, and preferably, are commercially available, and include pipecolic acid, thiazolidine carboxylic acid, dehydroproline, 3- and 4-methylproline, and 3,3-dimethylproline.

Essential amino acids in the parent polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for biological activity (i.e., antibacterial activity) to identify amino acid residues that are critical to the activity of the molecule. See also, Hilton et al., 1996, J. Biol. Chem. 271: 4699-4708. The biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309:59-64. The identities of essential amino acids can also be inferred from analysis of identities with polypeptides which are related to a polypeptide according to the invention.

Single or multiple amino acid substitutions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochem. 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide of interest, and can be applied to polypeptides of unknown structure.

In a preferred embodiment, the polypeptides of the invention are defensin polypeptides, preferably insect- or arthropod-defensin polypeptides.

### N-Terminal Extension

An N-terminal extension of the polypeptides of the invention may suitably consist of from 1 to 50 amino acids, preferably 2-20 amino acids, especially 3-15 amino acids. In one embodiment N-terminal peptide extension does not contain an Arg (R). In another embodiment the N-terminal extension comprises a kex2 or kex2-like cleavage site as will be defined further below. In a preferred embodiment the N-terminal extension is a peptide, comprising at least two Glu (E) and/or Asp (D) amino acid residues, such as an N-terminal extension comprising one of the following sequences: EAE, EE, DE and DD.

### Kex2 Sites

Kex2 sites (see, e.g., Methods in Enzymology, Vol 185, ed. D. Goeddel, Academic Press Inc. (1990), San Diego, Calif., “Gene Expression Technology”) and kex2-like sites are di-basic recognition sites (i.e., cleavage sites) found between the pro-peptide encoding region and the mature region of some proteins.

Insertion of a kex2 site or a kex2-like site have in certain cases been shown to improve correct endopeptidase processing at the pro-peptide cleavage site resulting in increased protein secretion levels.

In the context of the invention insertion of a kex2 or kex2-like site result in the possibility to obtain cleavage at a certain position in the N-terminal extension resulting in an antibacterial polypeptide being extended in comparison to the mature polypeptide shown in SEQ ID NO: 1.

### Fused Polypeptides

The polypeptides of the present invention also include fused polypeptides or cleavable fusion polypeptides in which another polypeptide is fused at the N-terminus or the C-terminus of the polypeptide of the invention or a fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

### Methods and Uses

The invention relates to the use of a polypeptide of the invention for treating intracellular bacterial infections in phagocytic cells, preferably macrophages. Accordingly, the polypeptides of the invention may be used in the preparation of veterinarian or human therapeutic agents or prophylactic agents for the treatment of intracellular bacterial infections in phagocytic cells, preferably macrophages. Intracellular bacterial infection means that the bacteria causing the infection are residing inside human or animal cells.

In a preferred embodiment, the intracellular bacterial infection is an intracellular Gram-positive bacterial infection; more preferably the intracellular bacterial infection is an intracellular Staphylococcus infection; most preferably the intracellular bacterial infection is an intracellular Staphylococcus aureus infection.

The polypeptides of the invention are useful for the treatment of Chronic Granulomatous Disease (CGD), which is a disease wherein cells of the immune system (phagocytic cells) have difficulty forming the reactive oxygen compounds (most importantly, the superoxide radical) used to kill ingested pathogens, such as Staphylococcus aureus. This leads to the formation of granulomata in many organs. Patients with CGD will suffer from recurrent bouts of infection due to the decreased capacity of their immune system to fight off disease-causing organisms partly residing intracellularly. Since the polypeptides of the invention are capable of exhibiting antibacterial activity intracellularly on pathogens ingested by phagocytic cells, they may be used for the manufacturing of a medicament for the treatment of Chronic Granulomatous Disease.

The polypeptides of the invention may be used in an amount sufficient to kill or inhibit growth of Staphylococcus sp., such as Staphylococcus aureus, residing inside human or animal cells.

Formulations of the polypeptides of the invention are administered to a host suffering from or predisposed to an intracellular bacterial infection in phagocytic cells, preferably macrophages. In an embodiment, the intracellular bacterial infection is caused by infection with Staphylococcus sp., such as Staphylococcus aureus.

Administration may be localized or systemic. Generally the dose of the antibacterial polypeptides of the invention will be sufficient to decrease the microbial population by at least 1 log, and may be by 2 or more logs of killing. The polypeptides of the present invention are administered at a dosage that reduces the microbial population while minimizing any side-effects. It is contemplated that the composition will be obtained and used under the guidance of a physician for in vivo use.

Various methods for administration may be employed. The polypeptide formulation may be given orally, or may be injected intravascularly, intramuscular, subcutaneously, peritoneally, by aerosol, opthalmically, intra-bladder, topically, etc. The dosage of the therapeutic formulation will vary widely, depending on the specific antibacterial polypeptide to be administered, the frequency of administration, the manner of administration, the clearance of the agent from the host, and the like. The initial dose may be larger, followed by smaller maintenance doses. Preferably the initial dose is at least 17 mg/kg. In many cases, oral administration will require a higher dose than if administered intravenously. The amide bonds, as well as the amino and carboxy termini, may be modified for greater stability on oral administration. For example, the carboxy terminus may be amidated.

### Formulations

The polypeptides of this invention can be incorporated into a variety of formulations for therapeutic administration. More particularly, the polypeptides of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the polypeptides can be achieved in various ways, including oral, buccal, rectal, parenteral, intraperitoneal, intradermal, transdermal, intracheal, etc., administration. The antibacterial polypeptides of the invention may be systemic after administration or may be localized.

The polypeptides of the present invention can be administered alone, in combination with each other, or they can be used in combination with other known compounds (e.g., perforin, anti-inflammatory agents, antibiotics, etc.) In pharmaceutical dosage forms, the polypeptides may be administered in the form of their pharmaceutically acceptable salts. The following methods and excipients are merely exemplary and are in no way limiting.

For oral preparations, the polypeptides can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The polypeptides can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The polypeptides can be utilized in aerosol formulation to be administered via inhalation. The polypeptides of the present invention can be formulated into pressurized acceptable propellants such as dichlorodifluoromethane, propane, nitrogen and the like.

Furthermore, the polypeptides can be made into suppositories by mixing with a variety of bases such as emulsifying bases or water-soluble bases. The polypeptides of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more polypeptides of the present invention. Similarly, unit dosage forms for injection or intravenous administration may comprise the polypeptide of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the antibacterial polypeptides of the invention is placed in proximity to the site of infection, so that the local concentration of active agent is increased relative to the rest of the body.

The term “unit dosage form”, as used herein, refers to physically discrete units suitable as unitary dosages for human and animal subjects, each unit containing a predetermined quantity of polypeptides of the present invention calculated in an amount sufficient to produce the desired effect in association with a pharmaceutically acceptable diluent, carrier or vehicle. The specifications for the unit dosage forms of the present invention depend on the particular polypeptide employed and the effect to be achieved, and the pharmacodynamics associated with the polypeptide in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific polypeptide, the severity of the symptoms and the susceptibility of the subject to side effects. Some of the specific polypeptides are more potent than others. Preferred dosages for a given polypeptide are readily determinable by those of skill in the art by a variety of means. A preferred means is to measure the physiological potency of a given polypeptide.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will normally be neutral or acidic lipids, such as cholesterol, phosphatidyl serine, phosphatidyl glycerol, and the like.

For preparing the liposomes, the procedure described by Kato et al., 1991, J. Biol. Chem. 266:3361 may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the antibacterial polypeptides of the invention may be formulated with other pharmaceutically active agents, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the antibacterial polypeptides of the invention, e.g., interferon gamma, tumor necrosis factor alpha, interleukin 12, etc.

### In Vitro Synthesis

The polypeptides of the invention may be prepared by in vitro synthesis, using conventional methods as known in the art. Various commercial synthetic apparatuses are available, for example automated synthesizers by Applied Biosystems Inc., Beckman, etc. By using synthesizers, naturally occurring amino acids may be substituted with unnatural amino acids, particularly D-isomers (or D-forms), e.g., D-alanine and D-isoleucine, diastereoisomers, side chains having different lengths or functionalities, and the like. The particular sequence and the manner of preparation will be determined by convenience, economics, purity required, and the like.

Chemical linking may be provided to various peptides or proteins comprising convenient functionalities for bonding, such as amino groups for amide or substituted amine formation, e.g., reductive amination, thiol groups for thioether or disulfide formation, carboxyl groups for amide formation, and the like.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

The present invention is further described by the following examples that should not be construed as limiting the scope of the invention.

### EXAMPLES

In the following Examples, the defensin polypeptide shown as SEQ ID NO: 1 is hereinafter referred to as “Defensin2114”; and the defensin polypeptide shown as SEQ ID NO: 2 is hereinafter referred to as “Plectasin”.

### Example 1

### In Vitro Study on the Intracellular Effect of Plectasin and Defensin2114 in THP-1 Macrophages

This Example presents an in vitro study of the intracellular antibacterial activity of Plectasin and Defensin2114 in infected THP-1 macrophages.

### Preparation of Bacterial Strain

For studies of Plectasin, two strains of S. aureus were applied: ATCC25923 (MSSA) and E33235 (MSSA, axil, clinical isolate from Statens Serum Institute, Denmark).

For studies of Defensin2114, two strains of S. aureus were used: ATCC25923 (MSSA), MRSA (clinical septicaemia isolate, Statens Serum Institute, ref. no. 1-15479) and VRSA2 (Pennsylvania HIP 11983).

Overnight culture of bacteria was opsonized for 45 min with human serum (AB positive, Lonza, USA). Afterwards the bacteria were tittered using a spectrophotometer (CECIL 2040) and THP-1 macrophages were counted and then infected with 4 bacteria/cell. THP-1 cells were cultured in RPMI 1640 medium supplemented with fetal calf serum.

The bacteria were incubated with cells for 1 hour at 37° C., CO₂ 5%, to allow phagocytosis.

### Washes

After 1 hour of incubation the infected cells were incubated 45 min with Gentamicin (50 micrograms/mL) to eradicate remaining non-phagocytosed bacteria. To remove Gentamicin after incubation cells were washed 3× with PBS. After the third wash, cells were re-suspended in RPMI 1640 medium (+10% fetal calf serum) and Plectasin or Defensin2114 was added at desired concentrations (0-128×MIC). One sample was incubated with Gentamicin at 1×MIC for control of intracellular growth. In studies of Defensin2114 the activity was compared to that of vancomycin and daptomycin.

Cells were incubated in 6-well plates for 24 hours at 37° C., CO₂ 5%.

After incubation cells were washed in PBS and subsequently re-suspended in sterile water to release intracellular bacteria.

### CFU Amount Determination

Samples were diluted regarding to the expected amount of CFU and spread on TSA plates and incubated at 35° C. overnight. After 24 hours incubation the CFU were counted.

### Cellular Protein Assay

The samples may be stored at −20° C. for up to one week. Samples were sonicated and proteins titered as described by Lowry et al. hereby obtaining results as CFU/mg protein. All samples were tested in triplicates.

### Data Analysis

In vitro intracellular dose-response studies: For analysis of dose-effect relationships, the Hill-equation (slope=1) was employed to calculate the maximal relative efficacy, Eₘₐₓ, static concentration (C_{static}) and goodness-of-fit. These parameters were determined using non-linear regression.

The Eₘₐₓ was defined as the log change in CFU counts between post-phagocytosis inoculum and 24 hour treatment, while the C_{static} was the concentration (in multiples of the MIC) resulting in no apparent bacterial growth (CFU identical to original inoculum).

### Results

Plectasin Versus Staphylococcus aureus ATCC25923 and E33235 after 24 Hours:

TABLE 1
Maximal effects, Eₘₐₓ (with confidence intervals, CI), and static
dose, C_{static}, from in vitro intracellular studies of Plectasin.

| | | Eₘₐₓ (CI) | C_{static} | |
|---|---|---|---|---|
| Bacteria | Antibiotic | (log CFU) | (×MIC) | R² |
| E33235 | Plectasin | −0.97 | 0.6 | 0.952 |
| | | (−1.78 to −0.76) | | |
| ATCC25923 | Plectasin | −1.35 | 0.6 | 0.882 |
| | | (−1.92 to −0.79) | | |

Defensin2114 Versus Staphylococcus aureus ATCC25923, MRSA (1-15479) and VRSA2 after 24 Hours in Comparison with Vancomycin and Daptomycin:

TABLE 2
Maximal effect, Eₘₐₓ (with 95% confidence intervals, 95%
CI), and static dose, C_{static}, from in vitro intracellular
studies of Defensin2114, daptomycin and vancomycin.

| | | Eₘₐₓ (95% CI) | C_{static} | |
|---|---|---|---|---|
| Bacteria | Antibiotic | (log CFU) | (×MIC) | R² |
| ATCC25923 | Defensin2114 | −1.51 | 0.8 | 0.901 |
| | | (−1.80 to −1.22) | | |
| | Daptomycin | −1.00 | 0.6 | 0.927 |
| | | (−1.25 to −0.76) | | |
| | Vancomycin | −0.64 | 2.9 | 0.823 |
| | | (−0.99 to −0.29) | | |
| MRSA | Defensin2114 | −0.93 | 1.1 | 0.868 |
| (1-15479) | | (−1.27 to −0.58) | | |
| | Daptomycin | −0.85 | 1.8 | 0.888 |
| | | (−1.13 to −0.57) | | |
| | Vancomycin | −0.66 | 6.2 | 0.895 |
| | | (−0.99 to −0.32) | | |
| VRSA2 | Defensin2114 | −0.22 | 2.3 | 0.907 |
| | | (−0.33 to −0.11) | | |
| | Daptomycin | −0.29 | 1.3 | 0.851 |
| | | (−0.44 to −0.16) | | |

Eₘₐₓ: Decrease in log CFU after 24 hours compared to original inoculum (T = 0 hours).
C_{static}: Concentration (in times the MIC) resulting in no apparent growth of bacteria.

### Statistical Analysis: One-Way ANOVA Analysis (Tukeys Test)

ATCC25923: Defensin2114 had significantly lower Eₘₐₓ value than both daptomycin and vancomycin.

MRSA: Defensin2114 had significantly lower Eₘₐₓ value than vancomycin (P<0.01).

VRSA: No difference in Eₘₐₓ value between Defensin2114 and daptomycin.

For both Plectasin and Defensin2114 it should be noted that a marked intracellular effect is reached at drug concentrations of 1-8×MIC. Above these levels no additional increase in activity is observed.

### Example 2

### In Vivo Study on the Intracellular Effect of Plectasin Against Staphylococcus aureus

This Example presents an in vivo study of the efficacy of Plectasin against intracellular Staphylococcus aureus E33235 in an animal model of infection (peritonitis), including assessment of both intra- and extracellular effect. Staphylococcus aureus E33235 is a clinically isolated strain obtainable from Statens Serum Institut, Denmark.

### Calculations for Dosing Regimens

MIC for Staphylococcus aureus E33235 was determined to 2 mg/L. From extrapolations from a kinetics study of doses from 4.25-34 mg/kg Plectasin, AUC and T>MIC were determined.

### Kinetics Study:

Animals were dosed from 4.25-34 mg/kg

At 4.25 mg/kg and 8.5 mg/kg, blood samples were taken after 5, 10, 20, 40, 60, 120 and 240 min.

At 17 mg/kg and 34 mg/kg, blood samples were taken after 5, 10, 20, 30, 40, 60, 120, 180 and 360 min.

The serum was separated and stored at −20° C. until analysis by LCMSMS using internal and external standards. Serum binding was calculated to 90%.

Pharmacokinetic parameters were determined through intrapolation from free serum concentrations using GraphPad Prism.

TABLE 3
Pharmacokinetic parameters

| Dose | 4.25 | 8.5 | 17 | 34 |
|---|---|---|---|---|
| AUC(free) | 1.901 | 3.955 | 8 | 13.25 |
| T > MIC(free) | 0 | 0.1204 | 2.334 | 5.548 |
| Peak | 1.61 | 2.32 | 4.95 | 5.99 |

TABLE 4
Dosing regimens in 24 hours PK/PD study with varying parameters

| Total | | | | | | |
|---|---|---|---|---|---|---|
| dose | Single | Doses | | | | |
| (mg/ | dose | in 24 | AUC/ | T > MIC | | Treatment |
| mouse) | (mg/kg) | hours | MIC | (h) | Cpeak | Regimen # |
| 1.20 | 34 | 1 | 2.638 | 6.63 | 5.99 | 1 |
| 0.60 | 17 | 1 | 1.611 | 3.96 | 4.95 | 2 |
| 0.30 | 8.5 | 1 | 0.9463 | 1.98 | 2.32 | 3 |
| 0.15 | 4.25 | 1 | 0 | 0.95 | 1.61 | 4 |
| 1.20 | 17 | 2 | 5.276 | 13.25 | 5.99 | 5 |
| 0.60 | 8.5 | 2 | 1.8926 | 3.96 | 2.32 | 6 |
| 3.60 | 34 | 3 | 7.914 | 19.88 | 5.99 | 7 |
| 0.45 | 4.25 | 3 | 0 | 2.85 | 1.61 | 8 |
| 1.20 | 8.5 | 4 | 3.7852 | 7.91 | 2.32 | 9 |
| 1.80 | 8.5 | 6 | 5.6778 | 11.87 | 2.32 | 10 |

### Experimental Day 1

Staphylococcus aureus E33235 was spread on 5% blood plates and incubated overnight at 37° C. at ambient air. A 10% w/v Mucin solution was prepared in NaCl. The solution was sterilized and pH adjusted to pH 7.0.

### Experimental Day 2

### Inoculum

Colonies of Staphylococcus aureus were suspended in saline to approx. 10⁸ CFU/mL (OD=0.13). A 1:1 dilution with the 10% Mucin stock solution was prepared making a solution of 5% Mucin and 5×10⁷ CFU/mL.

### Preparation of Solutions

A solution of 50 mg/mL lysostaphin was prepared in HBSS (Hanks Balanced Salt Solution).

### Inoculation of Mice

Two hours before treatment (T=0) the mice were inoculated i.p. with 0.5 mL Staphylococcus aureus suspension (with 5% Mucin).

### Treatment of Mice

The mice were treated s.c. with Plectasin-solutions at different time points as shown in Table 2. The injected dose volume was 0.6 or 0.3 mL/animal. The first dose was injected two hours after inoculation (T=2).

### Peritoneal Wash

At T=2 and T=6 hours control animals were sacrificed and peritoneal wash performed. 24 hours after first treatment, the remaining treated animals were sacrificed. Peritoneal wash was performed after sacrifice of animals by cervical dislocation.

### Separation Assay (Intra- and Extracellular Bacteria)

TABLE 5
Overview of samples

| Sample | Strain | Sampling time | Animal no. |
|---|---|---|---|
| 1-6 | S. aureus E 33235, controls | 2 hours after | Mouse 1-6 |
| | (no treatment) | treatment | |
| 7-12 | S. aureus E 33235, controls | 6 hours after | Mouse 7-12 |
| | (no treatment) | treatment | |
| 13-15 | S. aureus E 33235, treatment | 24 hours after | Mouse 13-15 |
| | regimen 1 | 1st treatment | |
| 16-18 | S. aureus E 33235, treatment | 24 hours after | Mouse 16-18 |
| | regimen 2 | 1st treatment | |
| 19-21 | S. aureus E 33235, treatment | 24 hours after | Mouse 19-21 |
| | regimen 3 | 1st treatment | |
| 22-24 | S. aureus E 33235, treatment | 24 hours after | Mouse 22-24 |
| | regimen 4 | 1st treatment | |
| 25-27 | S. aureus E 33235, treatment | 24 hours after | Mouse 25-27 |
| | regimen 5 | 1st treatment | |
| 28-30 | S. aureus E 33235, treatment | 24 hours after | Mouse 28-30 |
| | regimen 6 | 1st treatment | |
| 31-33 | S. aureus E 33235, treatment | 24 hours after | Mouse 31-33 |
| | regimen 7 | 1st treatment | |
| 34-36 | S. aureus E 33235, treatment | 24 hours after | Mouse 34-36 |
| | regimen 8 | 1st treatment | |
| 37-39 | S. aureus E 33235, treatment | 24 hours after | Mouse 37-39 |
| | regimen 9 | 1st treatment | |
| 40-42 | S. aureus E 33235, treatment | 24 hours after | Mouse 40-42 |
| | regimen 10 | 1st treatment | |
| 43 | S. aureus E 33235 | 24 hours after | Neg. control |
| | | 1st treatment | |

Negative control (cell free): Inoculum at 10⁸ CFU/mL of S. aureus E 33235 (OD=0.13) in saline was prepared just before the last separation assay of the day. The negative control was handled exactly as the test samples. Microscopy and cell-count of the negative control are not shown. Each sample was handled as described in Table 6.

TABLE 6
Procedure for handling of peritoneal samples.

A microscopy slide with 20 microliters of the sample is prepared.
20 micro-L of the sample is transferred to 5.0 mL of isotonic diluent and
the WBC-count is performed.
The sample is divided into two fractions: the majority of the sample
(1.5 mL) is transferred to a 2.0 mL Eppendorf test tube. The other fraction
(minimum 200 micro-L) is transferred to a Widal test tube for
quantification of the total load of bacteria in the sample (CFU-1) and
stored at −70° C. until quantified.
The fraction in the Eppendorf tube is centrifuged for 10 min at 300 g and
25° C.
After centrifugation, 200 micro-L of the supernatant is transferred to a
Widal test tube for quantification of the extracellular load of bacteria
(CFU-2) and stored at −70° C. until quantified.
The pellet is resuspended in 1.0 mL of HBSS with lysostaphin (15
micro-g/mL) and is incubated for 7 min at room temperature.
The sample is centrifuged for 10 min at 300 g and 25° C.
After centrifugation, 200 micro-L of the supernatant is transferred to a
Widal test tube for control of the extracellular kill (CFU-3) and stored
at −70° C. until quantified as described.
The pellet is resuspended in 2.0 mL of HBSS.
The sample is centrifuged for 10 min at 300 g and 25° C.
The pellet is resuspended in 2.0 mL of HBSS.
The sample is centrifuged for 10 min at 300 g and 25° C.
The pellet is resuspended in 2.0 mL of HBSS.
The sample is centrifuged for 10 min at 300 g and 25° C.
The pellet is resuspended in 2.0 mL of HBSS.
The sample is centrifuged for 10 min at 300 g and 25° C.
The pellet is resuspended in 1.5 mL cold sterile water, thoroughly whirl
mixed and incubated for 15 min at room temperature.
The sample is transferred to a Widal test tube for quantification of the
intracellular load of bacteria in the sample (CFU-4) and stored at −70° C.
until quantified.

### Experimental Day 3

### Reading/counting CFU.

### Results

CFU counts from peritoneal fluid from treated animals were compared to PK/PD parameters to assess the correlation between intra- and extracellular effect of Plectasin to Cpeak, T>MIC and AUC/MIC.

Correlation (Hills) between the most predictive PK/PD-parameter. Effect was determined as the decrease in CFU in the peritoneal fluid 24 hours after the first treatment. Parameters were estimated by multiple regression analysis.

The results showed pronounced correlation between Cpeak and 1^{st} dose (Cpeak/T>MIC) and effect.

### Correlation Coefficient (R2):

### Cpeak

| | | | |
|---|---|---|---|
| R2: | Total: 0.78 | Ex: 0.85 | Int: 0.80 |

### T>MIC (1^{st} Dose)

| | | | |
|---|---|---|---|
| R2: | Total: 0.83 | Ex: 0.85 | Int: 0.80 |

### Low Correlation was Observed Between Effect and T>MIC and AUC/MIC:

### T>MIC

| | | | |
|---|---|---|---|
| R2: | Total: 0.20 | Ex: 0.29 | Int: 0.08 |

### AUC/MIC

| | | | |
|---|---|---|---|
| R2: | Total: 0.24 | Ex: 0.34 | Int: 0.11 |

Plectasin showed both intra- and extracellular effect in the mouse peritonitis model.

### Max Δ log Decrease in CFU Intracellularly:

−1.25 (SD: 0.058) at b.i.d. treatment with 34 mg/kg.

### Max Δ log Decrease in CFU Extracellularly:

−2.55 (SD: 0.150) at b.i.d. treatment with 34 mg/kg.

These results indicate both intra- and extracellular effect of Plectasin in the murine peritonitis model. Size of 1^{st} dose seems to be crucial for effect. Preferably, a first dose of at least 17 mg/kg should be used.

### Example 3

### Using the HMM Files from the PFAM Database to Identify a Defensin

Sequence analysis using hidden markov model profiles (HMM profiles) may be carried out either online on the Internet or locally on a computer using the well-known HMMER freely available software package. The current version is HMMER 2.3.2 from October 2003.

The HMM profiles may be obtained from the well-known PFAM database. The current version is PFAM 16.0 from November 2004. Both HMMER and PFAM are available for all computer platforms from, e.g., Washington University in St. Louis (USA), School of Medicine (http://pfam.wustl.edu and http://hmmer.wustl.edu).

If a query amino acid sequence, or a fragment thereof, belongs to one of the following five PFAM families, the amino acid sequence is a defensin according to the present invention:

Defensin_beta or “Beta Defensin”, accession number: PF00711;

Defensin_propep or “Defensin propeptide”, accession number: PF00879;

Defensin_{—}1 or “Mammalian defensin”, accession number: PF00323;

Defensin_{—}2 or “Arthropod defensin”, accession number: PF01097;

Gamma-thionin or “Gamma-thionins family”, accession number: PF00304.

An amino acid sequence belongs to a PFAM family, according to the present invention, if it generates an E-value which is greater than 0.1, and a score which is larger or equal to zero, when the PFAM database is used online, or when the hmmpfam program (from the HMMER software package) is used locally.

When the sequence analysis is carried out locally using the hmmpfam program, it is necessary to obtain (download) the HMM profiles from the PFAM database. Two profiles exist for each family; xxx_ls.hmm for glocal searches, and xxx_fs.hmm for local searches (“xxx” is the name of the family). That makes a total of ten profiles for the five families mentioned above.

These ten profiles may be used individually, or joined (appended) into a single profile (using a text editor—the profiles are ASCII files) that could be named, e.g., defensin.hmm. A query amino acid sequence can then be evaluated by using the following command line:
hmmpfam -E 0.1 defensin.hmm sequence_file

wherein “sequence_file” is a file with the query amino acid sequence in any of the formats recognized by the HMMER software package.

If the score is larger or equal to zero (0.0), and the E-value is greater than 0.1, the query amino acid sequence is a defensin according to the present invention.

The PFAM database is further described in Bateman et al., 2004, “The Pfam Protein Families Database”, Nucleic Acids Research 32 (Database Issue): D138-D141.

1. A polypeptide having antibacterial activity, which comprises an amino acid sequence having at least 80% identity to the amino acid sequence of SEQ ID NO: 1.

2. The polypeptide of claim 1, which comprises an amino acid sequence having at least 85% identity to the amino acid sequence of SEQ ID NO: 1.

3. The polypeptide of claim 1, which comprises an amino acid sequence having at least 90% identity to the amino acid sequence of SEQ ID NO: 1.

4. The polypeptide of claim 1, which comprises an amino acid sequence having at least 95% identity to the amino acid sequence of SEQ ID NO: 1.

5. The polypeptide of claim 1, which comprises an amino acid sequence having at least 97% identity to the amino acid sequence of SEQ ID NO: 1.

6. A method for the treatment of an intracellular bacterial infection in a phagocytic cell, comprising administering to a human or animal in need of such treatment a polypeptide of claim 1 in an effective amount for the treatment of the intracellular bacterial infection in a phagocytic cell.

7. The method of claim 6, wherein the phagocytic cell is a macrophage.

8. The method of claim 6, wherein the bacterial infection is a Staphylococcus aureus infection.

9. The method of claim 6, wherein the treatment includes a first dosage of the polypeptide of at least 17 mg/kg.

10. A method for the treatment of Chronic Granulomatous Disease, comprising administering to a human or animal in need of such treatment a polypeptide of claim 1 in an effective amount for the treatment of Chronic Granulomatous Disease.
